(19) United States
(12) Patent Application Publication
(10) Pub. No.: US 2026/0157855 A1
(43) Pub. Date: Jun. 11, 2026

(54) Knee Arthroplasty System for Balance in Full Range of Flexion

(71) Applicant: NEWTON-WELLESLEY HOSPITAL, Newton, MA (US)

(72) Inventors: Guoan LI, Newon, MA (US); Hany BEDAIR, Newton, MA (US); Timothy FOSTER, Newton, MA (US)

(21) Appl. No.: 18/708,041

(22) PCT Filed: Nov. 8, 2022
(86) PCT No.: PCT/US2022/049207

§ 371 Date: May 7, 2024

Related U.S. Application Data
(60) Provisional application No. 63/276,780, filed on Nov. 8, 2021.

(51) Int. Cl. A61F 2/38; A61F 2/30

(52) U.S. Cl. CPC A61F 2/3859; A61F 2/30942; A61F 2/389; A61F 2002/30948

(57) ABSTRACT

A knee joint prosthesis comprises a femoral component including a medial femoral condyle having an external surface including a first curved surface section, wherein the first curved surface section of the external surface of the medial femoral condyle has an arcuate shape defined by a radius from a medial femoral condyle IsoHeight Point to points on the first curved surface section of the external surface of the medial femoral condyle, and the femoral component further includes a lateral femoral condyle having an external surface including a first curved surface section of the external surface of the lateral femoral condyle, and the first curved surface section of the external surface of the lateral femoral condyle has an arcuate shape defined by a radius from a lateral femoral condyle IsoHeight Point to points on the first curved surface section of the external surface of the lateral femoral condyle.

### CROSS-REFERENCE TO RELATED APPLICATIONS

This application is based on, claims benefit of, and claims priority to U.S. Application No. 63/276,780 filed on Nov. 8, 2021, which is hereby incorporated by reference herein in its entirety for all purposes.

### STATEMENT REGARDING FEDERALLY SPONSORED RESEARCH

Not applicable.

### BACKGROUND OF THE INVENTION

### 1. Field of the Invention

This invention relates to a knee prostheses and methods for treating disease and trauma affecting the knee.

### 2. Description of the Related Art

Disease and trauma affecting the articular surfaces of the knee joint are commonly treated by surgically replacing the ends of the femur and tibia with prosthetic femoral and tibial implants, and, in some cases, replacing the patella with a patella component. Such surgeries are sometimes referred to as total knee arthroplasty (TKA). In TKA surgery, a surgeon typically affixes two prosthetic components to the subject's bone structure; a first to the subject's femur and a second to the subject's tibia. These components are typically known as the femoral component and the tibial component, respectively.

The femoral component is placed on a subject's distal femur after appropriate resection of the femur. The femoral component is usually metallic, having a highly polished outer condylar articulating surface. A common type of tibial component includes a top surface (plateau) that generally conforms to the subject's resected proximal tibia. The bottom surface of the tibial component also usually includes a stem that extends at an angle to the plateau in order to extend into a surgically formed opening in the tibial intramedullary canal. Two common designs of the tibial component exist. In one design, the tibial component is monolithic (single piece) and made of plastic/polymeric material. In another design, a plastic or polymeric (often ultra-high molecular weight polyethylene) tray is affixed on top of a tibial base plate which includes the stem and is usually made of metal. The top surface of the tibial component provides a surface against which the femoral component condylar portion articulates, i.e., moves in gross motion corresponding generally to the motion of the femur relative to the tibia.

Conventional knee prostheses have been developed without accounting for the full range of kinetics of active knee movement. A typical knee joint without a knee prosthesis is illustrated in FIG. 1A. The femur and tibia are shown with healthy femur and tibia cartilage. A portion of a prior art knee prosthesis 100 is shown in FIGS. 1B-1C. The knee prosthesis 100 is provided and is designed to replace at least a portion of a knee joint (the right knee is shown) between the distal end of a femur and the proximal end of a tibia. A mirror image (not shown) of knee prosthesis 100 will replace at least a portion of a left knee between the distal end of a femur and the proximal end of a tibia. As shown, the knee prosthesis 100 can include a tibial component 200 for mounting to a proximal end of a resected tibia, or to another prosthesis element such as a tibia base 260 that is mated to a resected tibia, and a femoral component 300 for mounting to a distal end of a resected femur. The tibial component 200 is shown in more detail in FIGS. 2 and 5. The tibial component 200 can be constructed in various manners and out of various materials. The tibial component 200 can be machined, cast, forged or otherwise constructed as a one-piece integral unit, or a two-piece unit, out of a medical grade, physiologically acceptable material such as polyethylene (e.g., high molecular weight polyethylene and/or vitamin E containing high molecular weight polyethylene) or the like, in various sizes to fit a range of typical subjects, or it can be custom designed for a specific subject based on data provided by a surgeon after physical and radiography examination of the specific subject. As noted above, the tibial component 200 can be configured for use with a tibial base 260. In other embodiments, the tibial component 200 can be used without a tibial base 260, with appropriate modifications known to those skilled in the art, and can be implanted directly on a resected tibia. In some embodiments, the tibial component 200 can have asymmetric medial 220 and lateral compartments 210, which interact with asymmetric medial and lateral femoral condyles of the femoral component 300 to restore normal/physiologic knee motion characterized by: (a) axial rotation of the femur about an overall medially located pivot point, and (b) posterior motion of the femur center, with knee flexion. In some embodiments for partial knee replacement, a medial compartment 220 and a lateral compartment 210 can be used alone and/or in combination with a center portion and/or in combination with the tibial base 260. An overall size of the tibial component 200, having edge 225, can vary depending on the size of the subject. Looking at FIG. 5, the lateral compartment 210 includes a posterior lip 330 and an anterior lip 335. The medial compartment 220 can also have similar posterior lip and anterior lips. FIG. 3 is a side view of the distal portion of a human femur, and FIG. 4 is a side view of the femoral prosthesis 300, displaying the profiles of the medial and lateral femoral condyles.

While TKA is a highly successful surgical treatment option for severe knee joint disease such as osteoarthritis and rheumatoid arthritis, several studies have shown that current TKA implants do not restore the motion of the knee to the normal/healthy state, thus limiting subject function following surgery. Typically, the active (as opposed to passive when muscles are relaxed) range of knee flexion following TKA is limited to less than 115 degrees, whereas the healthy knee is capable of bending up to 160 degrees. Increased range of knee motion is required for activities like squatting and kneeling, which are particularly important for subjects of certain ethnic and religious groups, as well as certain occupations and leisure activities. In addition to limited range of motion, complications, particularly of the patellofemoral joint, including chronic pain, patellar subluxation, patellar tilt, patellar dislocation and patellar component loosening have also been observed in 1-20% of TKA subjects.

These limitations of TKA have in part been related to the inability of existing designs to replicate in vivo knee joint kinematics, including the kinematics of the femur relative to the tibia (tibiofemoral kinematics) and the patella relative to the femur (patellofemoral kinematics). The tibiofemoral kinematics following TKA are characterized by reduced posterior femoral translation and reduced internal tibial rotation, compared to normal knees. In addition, unexpected anterior femoral translation has been frequently noted in knees with TKA. Current TKA designs have also been shown to have abnormal patellofemoral kinematics. For example, studies have shown more superior patellofemoral contact, inconsistent patellar tracking, patellofemoral separation, and higher patellar tilt angles in TKA compared to normal knees.

Accordingly, there remains a need for improved knee prostheses and methods for treating disease and trauma affecting the knee.

### SUMMARY OF THE INVENTION

The present invention meets the foregoing needs by providing improved TKA systems and methods that more closely resemble features of native knee anatomy and motion patterns, and greatly reduce unexpected anterior and posterior femoral translation.

In one aspect, the disclosure provides a knee joint prosthesis comprising a femoral component including an internal non-articulating bone-engaging surface configured to be connected to a resected distal end of a femur, the femoral component including a medial femoral condyle having an external surface configured to articulate with a bearing surface of a tibial component, and the external surface of the medial femoral condyle including a first curved surface section. Additionally, the first curved surface section of the external surface of the medial femoral condyle has an arcuate shape defined by a radius from a medial femoral condyle IsoHeight Point to points on the first curved surface section of the external surface of the medial femoral condyle.

In one embodiment, the femoral component further includes a lateral femoral condyle having an external surface configured to articulate with a bearing surface of a tibial component, and the external surface of the lateral femoral condyle including a first curved surface section of the external surface of the lateral femoral condyle. Additionally, the first curved surface section of the external surface of the lateral femoral condyle has an arcuate shape defined by a radius from a lateral femoral condyle IsoHeight Point to points on the first curved surface section of the external surface of the lateral femoral condyle.

In another aspect, the disclosure provides a femoral component including an internal non-articulating bone-engaging surface configured to be connected to a resected distal end of a femur, the femoral component including a lateral femoral condyle having an external surface configured to articulate with a bearing surface of a tibial component, the external surface of the lateral femoral condyle including a first curved surface section of the external surface of the lateral femoral condyle. Additionally, the first curved surface section of the external surface of the lateral femoral condyle has an arcuate shape defined by a radius from a lateral femoral condyle IsoHeight Point to points on the first curved surface section of the external surface of the lateral femoral condyle.

In one embodiment, the first curved surface section of the external surface of the medial femoral condyle can transition to a posterior surface section of the external surface of the medial femoral condyle, and the posterior surface section of the external surface of the medial femoral condyle can have a shape at least partially defined by a posterior section radius having a length less than a length of the radius from the medial femoral condyle IsoHeight Point to the points on the first curved surface section of the external surface of the medial femoral condyle.

In another embodiment, the first curved surface section of the external surface of the medial femoral condyle can transition to an anterior surface section of the external surface of the medial femoral condyle, and the anterior surface section of the external surface of the medial femoral condyle can have a shape at least partially defined by an anterior section radius having a length greater than the length of the radius from the medial femoral condyle IsoHeight Point to the points on the first curved surface section of the external surface of the medial femoral condyle.

In another embodiment, the first curved surface section of the external surface of the lateral femoral condyle can transition to a posterior surface section of the external surface of the lateral femoral condyle, and the posterior surface section of the external surface of the lateral femoral condyle can have a shape at least partially defined by a posterior section radius having a length less than a length of the radius from the lateral femoral condyle IsoHeight Point to the points on the first curved surface section of the external surface of the lateral femoral condyle.

In another embodiment, the first curved surface section of the external surface of the lateral femoral condyle can transition to an anterior surface section of the external surface of the lateral femoral condyle, and the anterior surface section of the external surface of the lateral femoral condyle can have a shape at least partially defined by an anterior section radius having a length greater than the length of the radius from the lateral femoral condyle IsoHeight Point to the points on the first curved surface section of the external surface of the lateral femoral condyle.

In another embodiment, the knee joint prosthesis can include a tibial component, and the femoral component and the tibial component can articulate so as to maintain substantially constant condylar heights at a medial side and a lateral side of the knee through the full range of flexion. The heights of the lateral femoral condyle and the medial femoral condyle can be different to articulate on corresponding surfaces of the tibial component. A substantially constant condyle height at a medial side and/or a lateral side of the knee can be achieved by minimizing vertical height changes of the medial femoral condyle IsoHeight Point or the lateral femoral condyle IsoHeight Point through coordinated articulation of anterior-posterior tibial slopes with the medial femoral condyle or the lateral femoral condyle.

In another embodiment, the medial femoral condyle and the lateral femoral condyle can be in an asymmetric configuration. The knee joint prosthesis can include a tibial component, and a surface of the tibial component can include both a medial and lateral surface having the same height and each having an articular surface substantially asymmetric with each other for at least portions thereof. The surface of the tibial component may include both a medial and lateral surface having a deeper medial tibial surface height than a lateral tibial surface height and each having an articular surface substantially asymmetric with each other for at least portions thereof.

In another embodiment, the knee can further comprise design elements for posterior stabilization, cruciate retaining, bi-cruciate stabilization and/or bi-cruciate retaining functionality. The prosthesis may exhibit no mid-range articulation transition zone to at least minimize mid-range instability. Additionally, no sudden change in knee loading may be promoted through a full range of flexion, and the full range of flexion can comprise from approximately −10 degrees to 130 degrees. The prosthesis can further comprise a material selected from the group consisting of 3D printable polymers, polyethylene, cross-link polyethylene, polyether ether ketone (PEEK), titanium, titanium alloy, tantalum, cobalt chrome alloy, stainless steel, and ceramics, and the prosthesis can maintain medial and lateral ligament tension balances through a full range of motion without overstretching the soft-tissue in the knee.

In another aspect, the disclosure provides a method comprising determining from anatomical and kinematic analysis an appropriate medial femoral condyle IsoHeight Point and an appropriate lateral femoral condyle IsoHeight Point for a subject; and selecting or fabricating for implantation a knee prosthesis.

In the method, determining can comprise acquiring images of the subject's knee with or without motion thereof and calculating from the acquired images appropriate dimension specifications for the knee joint prosthesis. The method can further comprise surgically implanting the knee joint prosthesis into the subject's knee through manual, navigational, or robotic methods.

In another aspect, the disclosure provides a method for manufacturing a knee joint prosthesis, the method comprising forming a femoral component including an internal non-articulating bone-engaging surface configured to be connected to a resected distal end of a femur, wherein the femoral component includes a medial femoral condyle having an external surface configured to articulate with a bearing surface of a tibial component, the external surface of the medial femoral condyle including a first curved surface section, wherein the first curved surface section of the external surface of the medial femoral condyle has an arcuate shape defined by a radius from a medial femoral condyle IsoHeight Point to points on the first curved surface section of the external surface of the medial femoral condyle.

In the method, the medial femoral condyle IsoHeight Point can be a medial anatomic location of a medial femoral condyle determined by: (i) creating from medical images, a model of a knee joint of a subject including a femur and a tibia having ends to be replaced by a knee joint prosthesis, (ii) orienting a longitudinal axis on the tibia of the model, (iii) placing a tibial plane on the model at a proximal end of the tibia of the model, the tibial plane being oriented within 0° to 10° of normal through the longitudinal axis of the tibia of the model, (iv) rotating the femur of the model through a full range of flexion, and (v) choosing the medial anatomic location to be a point in which a least amount of change of condyle height occurs throughout the full range of flexion from the tibial plane. The lateral femoral condyle IsoHeight Point can be a lateral anatomic location of a lateral femoral condyle determined by: (i) creating from medical images, a model of a knee joint of a subject including a femur and a tibia having ends to be replaced by a knee joint prosthesis, (ii) orienting a longitudinal axis on the tibia of the model, (iii) placing a tibial plane on the model at a proximal end of the tibia of the model, the tibial plane being oriented within 0° to 10° of normal through the longitudinal axis of the tibia of the model, (iv) rotating the femur of the model through a full range of flexion, and (v) choosing the lateral anatomic location to be a point in which a least amount of change of condyle height occurs throughout the full range of flexion from the tibial plane.

It is an advantage of the invention to provide systems and methods of total knee arthroplasty to maintain the native femoral condyle height along the flexion path of the knee to maintain the medial and lateral compartment balance of the knee. To keep the physiological condyle height, the tensions of the surrounding soft tissues such as medial and lateral collateral ligaments will be maintained.

It is another advantage of the invention to provide ways of avoiding tightness of the knee at high flexion and therefore, enhance the flexion capability of the knee since the design integrates the femoral condyle and tibial plateau surface design together using the physiological IsoHeight concept.

It is another advantage of the invention to provide systems and methods of easing gap/soft tissue balance along a flexion path of the knee, improving mid-range instability, reducing overstuffing at high flexion, enhancing natural knee feeling, and improving surgery longevity. Thus, the systems and methods described herein provide stability to the knee at low flexion, mid-flexion, and high flexion.

These and other features, aspects and advantages of various embodiments of the present invention will become better understood with regard to the following description, appended claims, and accompanying Figures.

### BRIEF DESCRIPTION OF THE DRAWINGS

FIG. 1A is a perspective view of a normal human knee.

FIG. 1B is a perspective view of a prior art exemplary knee prosthesis.

FIG. 1C is a perspective view of one embodiment of a femoral component and a tibial component of the knee prosthesis of FIG. 1B.

FIG. 2 is a top view of the tibial component of FIG. 1C.

FIG. 3 is a side view of the distal portion of a human femur.

FIG. 4 is a side view of the femoral prosthesis of the knee prosthesis of FIG. 1B, displaying the profiles of the medial and lateral femoral condyles.

FIG. 5 is a cross-sectional view of the tibial component of FIG. 2 taken along line 5-5 of FIG. 2 illustrating of the curvature of the lateral tibial compartment.

FIG. 6 is a perspective view of a 3D model of a human femur and human tibia.

FIG. 7 is a side view of virtual anatomic location points overlayed on a femoral condyle portion of a human knee joint.

FIG. 8 is a comparison of height changes of different virtual anatomic location points on medial and lateral femoral condyles of a human femur across a range of knee flexion angles.

FIG. 9 is a side view of a femoral and tibial component of a knee prosthesis traveling through a range of knee flexion angles.

FIG. 10 is an illustration of three curved surface sections that form one embodiment of a femoral condyle.

FIG. 11 is an illustration of the three curved surface sections of FIG. 10 travelling through a range of knee flexion angles.

FIG. 12 is a chart illustrating the difference in quadriceps force experienced by a knee during flexion between an IsoHeight prosthesis and a multi-radii prosthesis with a corresponding force body diagram.

FIG. 13 is a chart illustrating the difference in contact force experienced by a knee during flexion between an IsoHeight prosthesis and a multi-radii prosthesis with a corresponding force body diagram.

FIG. 14 is a chart illustrating the difference in shear force experienced by a knee during flexion between an IsoHeight prosthesis and a multi-radii prosthesis with a corresponding force body diagram.

FIG. 15 is a side view of an illustration of medial and lateral IsoHeight prostheses overlayed on a human femoral condyle.

FIG. 16 in panel (A) is a perspective view of a 3D knee model and coordinate systems used to measure femoral condyle heights during knee flexion; in panel (B) is a chart illustrating the trans-epicondylar axis (TEA), geometric-center axis (GCA), and IsoHeight axis points on a medial femoral condyle disk in the sagittal plane; and in panel (C) is a chart illustrating the TEA, GCA, and IsoHeight axis points on a lateral femoral condyle disk in the sagittal plane.

FIG. 17 is a comparison of medial and lateral femoral condyle heights during knee flexion measured using the TEA, GCA, and IsoHeight axis of panels (B) and (C) of FIG. 16.

FIG. 18 in panel (A) is a side view of medial and lateral femoral condyles designs with even medial and lateral tibial surfaces according to one embodiment of the present disclosure, and in panel (B) is a side view of medial and lateral femoral condyles designs with uneven medial and lateral tibial surfaces according to one embodiment of the present disclosure.

FIG. 19 is a side view of even medial (Medial-E), uneven medial (Medial-U), and lateral femoral condyles each including three curved surface sections according to one embodiment of the present disclosure.

FIG. 20 is an array of side views of even medial (Medial-E), uneven medial (Medial-U), and lateral femoral condyles with highlighted tibial lip engaging portions rotating through a knee flexion range.

FIG. 21 is an array of side views of even medial (Medial-E), uneven medial (Medial-U), and lateral femoral condyles rotating through a knee flexion range.

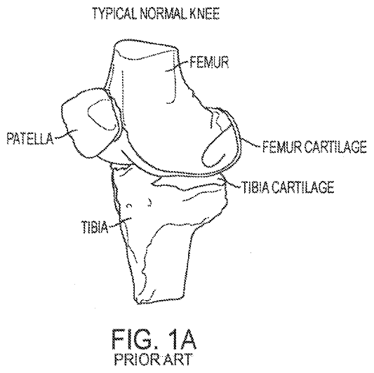

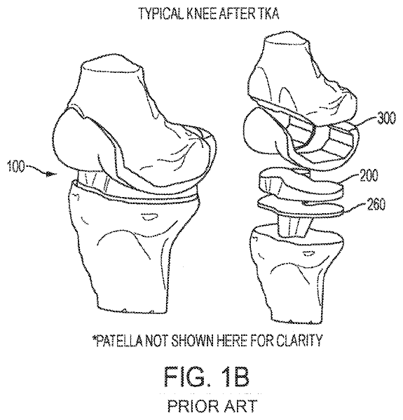

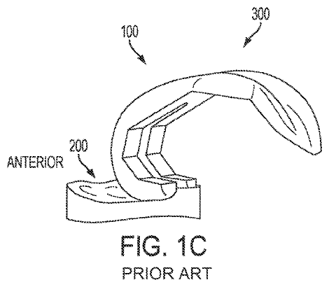

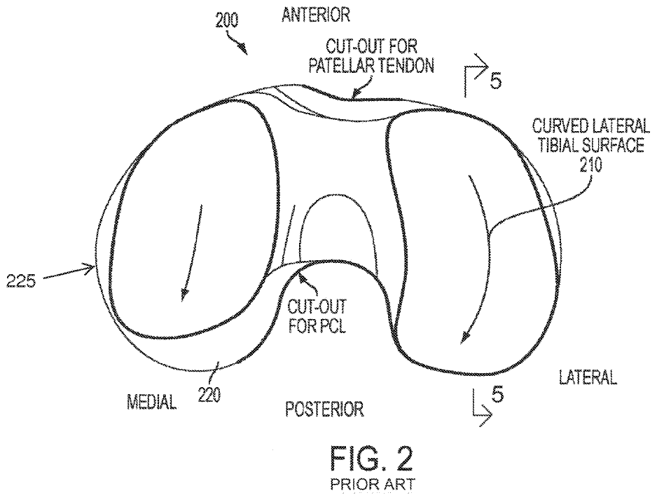

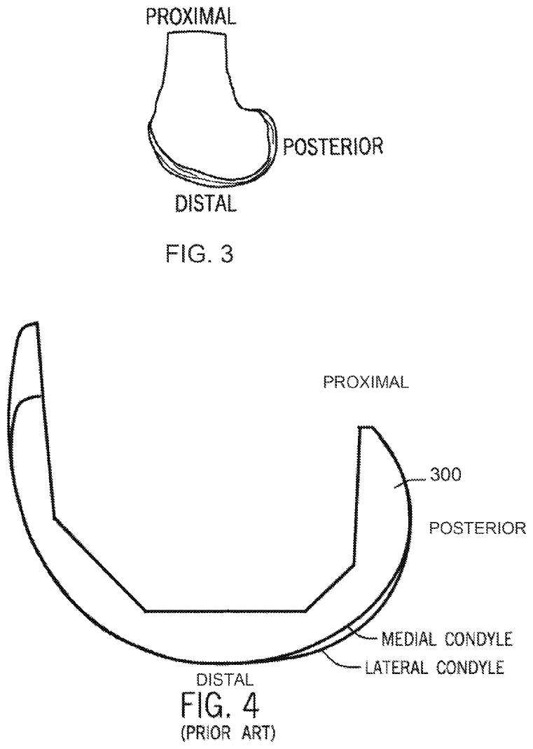

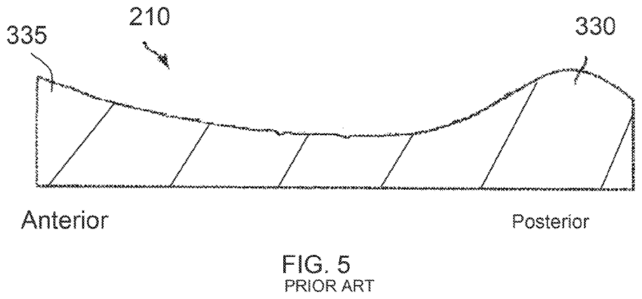

Like reference numerals will be used to refer to like parts from Figure to Figure in the following detailed description.

### DETAILED DESCRIPTION OF THE INVENTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

IsoHeight Point Definition: As used herein, the term “IsoHeight Point” refers to an anatomic location of a medial femoral condyle or a lateral femoral condyle determined by: (i) creating from medical images, a 3D model of a knee joint of a subject including a femur and a tibia having ends to be replaced by a knee joint prosthesis, (ii) orienting a longitudinal axis on the tibia of the model, (iii) placing a tibial plane on the model at a proximal end of the tibia of the model, the tibial plane being oriented within 0° to 10° of normal through the longitudinal axis of the tibia of the model, preferably within 5° to 7° of normal through the longitudinal axis of the tibia of the model, (iv) rotating the femur of the model through a full range of flexion, (v) choosing the anatomic location to be a point in which a least amount of change of femoral condyle height from the tibial plane occurs throughout the full range of flexion.

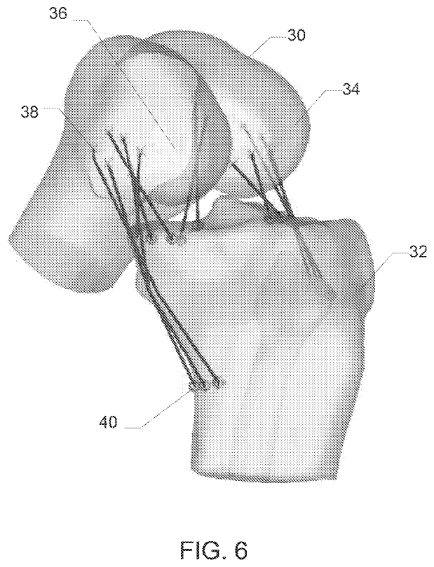

Referring now to FIG. 6, there is shown a perspective view of a 3D model of a human femur 30 and a human tibia 32. The 3D model is constructed using medical imaging techniques, i.e., X-ray computed tomography (CT), computed axial tomography (CAT), magnetic resonance imaging (MRI), or positron emission tomography (PET). In order to find an IsoHeight Point on a medial or lateral femoral condyle 34, 36, knee positions across a range of knee flexion angles are captured using advanced imaging technique, including but not limited to the dual fluoroscopic imaging system (DFIS). A DFIS is composed of two orthogonally positioned fluoroscopes. It can capture knee position simultaneously from two directions using paired images. In some aspects, the modeled femur 30 and tibia 32 are isolated and rotated to match the knee positions captured on paired fluoroscopic images through a full range of knee flexion angles, such as from −10° to 160°, 0° to 150°, 0° to 120°, or 0° to 110°. The knee positions in space are reproduced by a series of 3D knee models. Additionally, femoral reference points 38 are attached to corresponding tibial reference points 40 to model connective tissue between the femur and the tibia, i.e., muscles, ligaments, and tendons.

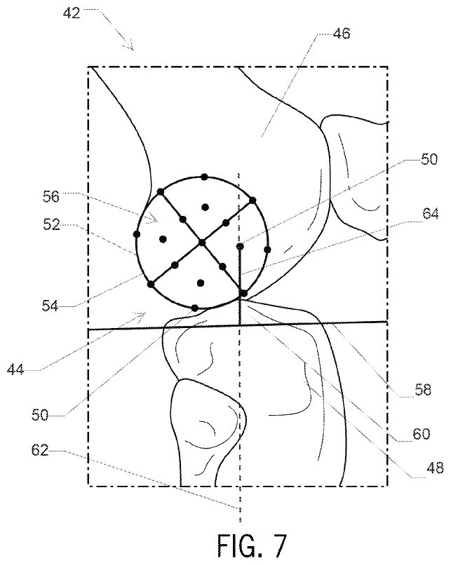

Referring now to FIG. 7, a side view of a medically obtained image 42 of a knee joint 44 including a femur 46, a tibia 48, and overlayed virtual anatomic location points 50 is illustrated. A reference circle 52 corresponding to a femoral condyle circle of rotation is overlayed onto the medically obtained image 42. In some instances, the points 50 are arranged on an outer circumference 54 of the circle 52 and concentrically within an area 56 of the circle 52, or the points 50 are arranged in a different geometric shape within the area 56 of the circle 52. In some aspects, the points 50 are arranged symmetrically with respect to the area 56 of the circle 52. A tibial plane 58 is then overlayed onto the medically obtained image 42 on a tibial surface 60 of the tibia 48. The tibial plane 58 is oriented within 0° to 10° of normal through a longitudinal axis 62 of the tibia 48, preferably within 5° to 7° of normal through the longitudinal axis 62 of the tibia 48. A condylar height 64 is defined as a distance measured between the tibial plane 58 and each of the virtual anatomic location points 50 along a direction normal to the tibial plane 58 for each point. An individual condylar height 64 exists for each anatomic location point 50. For example, an initial height for each anatomic location point 50 is measured as the condylar height at full extension position. As the femur 46 is rotated through the full range of knee flexion angles, the condylar height 64 of each anatomic location point 50 is continuously measured and recorded.

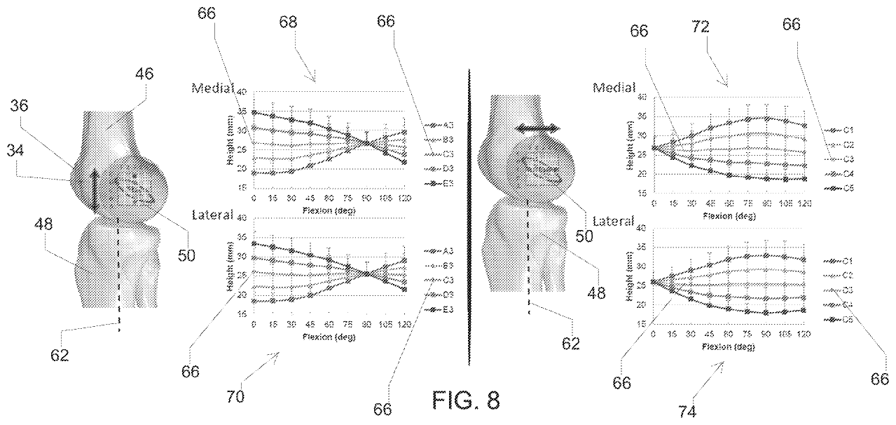

Referring now to FIG. 8, a comparison of condyle location changes for medial and lateral femoral condyle 34, 36 is illustrated. Vertical height is defined as a length measured from the tibial plane 58 to the virtual anatomic location points 50 along a direction perpendicular to the tibial plane 58 of the tibia 48. Horizontal length is defined as a length measured between a first position of each virtual anatomic location point 50 and a second position of each virtual anatomic location point 50 along a direction normal to the longitudinal axis 62 of the tibia 48. In some aspects, between 3 and 100, 3 and 50, 3 and 25, 3 and 15, 3 and 10, or 3 and 5 virtual anatomic location points 50 are used. As the femur 46 is rotated through a full range of knee flexion, the vertical height and horizontal length of each anatomic location point 50 are measured and recorded. The virtual anatomic location point 50 with the least amount of vertical height change along the flexion path is used as the IsoHeight Point 66. In some aspects, the IsoHeight Point 66 is also the virtual anatomic location point 50 with the least amount of horizontal length change, or the horizontal length change is not considered. In some aspects, the medial and lateral femoral condyle 34, 36 have different IsoHeight Points with different vertical height change profiles.

Example medial and lateral vertical height change charts 68, 70 describing the change in vertical height over knee flexion angles from 0° to 120° are illustrated in FIG. 8. In this example, the IsoHeight Point 66 was found to be virtual anatomic location point C3 for the medial femoral condyle 34 and lateral femoral condyle 36 since point C3 exhibited the least change in vertical height over the range of knee flexion. Additionally, example medial and lateral horizontal length change charts 72, 74 describing the change in horizontal length over knee flexion angles from 0° to 120° are illustrated in FIG. 8. In this example, the IsoHeight Point 66 was found to be virtual anatomic location point C3 for the medial femoral condyle 34 and lateral femoral condyle 36 since point C3 exhibited the least change in horizontal length over the range of knee flexion.

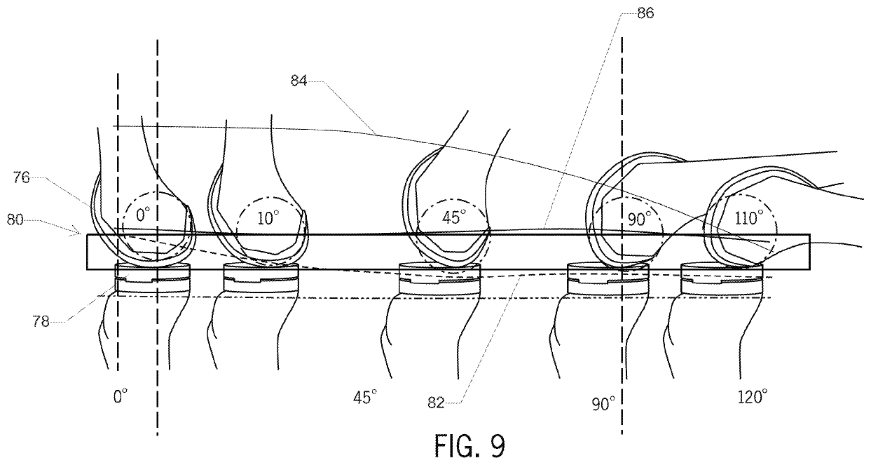

Referring now to FIG. 9, a side view of a femoral component 76 and tibial component 78 of a knee prosthesis 80 traveling through a range of knee flexion angles is illustrated. Traditionally, TKA surgery is performed using either a geometric center axis (GCA) 82 or a trans-epicondylar axis (TEA) 84 to measure condylar height. However, rotating a femur through a full range of flexion angles causes both the GCA 82 and TEA 84 to decrease in height at mid-flexion and high-flexion ranges. Using an IsoHeight axis 86 results in substantially constant condyle height and prevents overstuffing or laxation of the femoral component 76 that places undue strain on the knee. Thus, one advantage of the present disclosure over traditionally used TKA techniques is improved condyle height stability at mid-flexion and high-flexion angles.

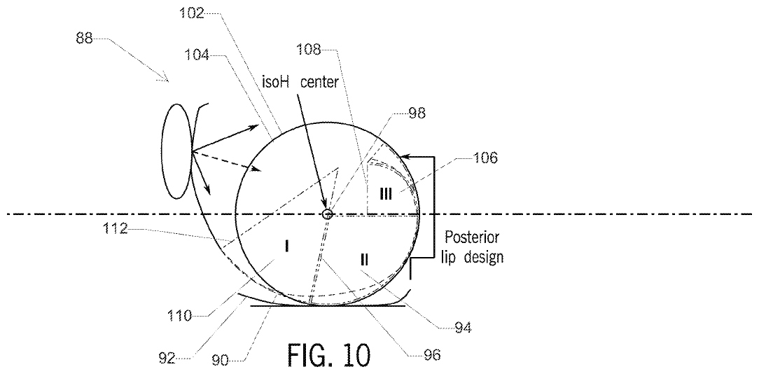

Referring now to FIG. 10., three curved surface sections that form one embodiment of a femoral condyle 88 are illustrated. The femoral component 76 includes an internal non-articulating bone-engaging surface configured to be connected to a resected distal end of a femur. The femoral component 76 further includes a medial femoral condyle 34 that has an external surface 90 configured to articulate with a bearing surface 92 of a tibial component, and the external surface 90 of the medial femoral condyle 34 includes a first curved surface section 94 (which includes label II). The first curved surface section 94 of the external surface 90 of the medial femoral condyle 34 has an arcuate shape defined by a radius 96 from a medial femoral condyle IsoHeight Point 98 to points on the first curved surface section 94 of the external surface of the medial femoral condyle 34.

An IsoHeight Point 98 exists at the center of an IsoHeight circle 102 that defines a circle of rotation of the femoral condyle 88. The first curved surface section 94 has a constant radius of curvature 96 from which the IsoHeight circle 102 extends such that the IsoHeight circle 102 and the first curved surface section 94 share a common radius 96. In some aspects, the first curved surface section 94 extends only along a portion of the IsoHeight circle 102, i.e., between 5% and 50%, 10% and 40%, 20% and 30%, 25% and 30%, or 30 and 35% of an outer circumference 104 of the IsoHeight circle 102. The first curved surface section 94 is configured to articulate with the bearing surface 92 of the tibial component along a flexion range of mid-flexion between 0 and 90 degrees or larger. In some embodiments, a first curved surface section 94 exists for the lateral femoral condyle 36, or each the medial femoral condyle 34 and the lateral femoral condyle 36. The first curved surface section 94 of the medial femoral condyle 34 has a radius of curvature of about 25 mm and the first curved surface section 94 of the lateral femoral condyle 36 has a radius of curvature of about 22 mm. As will be appreciated by those skilled in the art, the radius 96 of the first curved surface section 94 varies between the medial femoral condyle 34 and the lateral femoral condyle 36 depending on the size needed, and the radius 96 of the first curved surface section 94 can be in the range of 12 mm to 40 mm, 15 mm to 34 mm, 20 mm to 28 mm, or other ranges.

Still referring to FIG. 10, the first curved surface section 94 transitions to a posterior surface section 106 (which includes label III) of the external surface 90 of the medial or lateral femoral condyle 34, 36. The posterior surface section 106 has a shape at least partially defined by a posterior radius 108 having a length less than a length of the radius 96 from the IsoHeight Point 98 to the points of the first curved surface section 94 of the external surface 90 of the medial or lateral femoral condyle 34, 36. The posterior radius 108 of the posterior surface section 106 allows for stable, low stress contact even in very deep flexion, i.e., 150°. In some embodiments, the posterior surface section 106 has a reduced radius 108 corresponding to a more anatomic geometry that leads to an upward sloping tibial lip 95. This femoral geometry interacts with the tibial component ramp to prevent premature posterior shift of the femur. The posterior radius 108 simulates native posterior-proximal condyle anatomy and provides a recessed surface along the IsoHeight circle 102 which is supplemented by the upward sloping tibial lip, thereby maintaining the IsoHeight character of the femoral component 76 and constant condylar height. In some aspects, the posterior surface section 106 exists for each the medial or lateral femoral condyle 34, 36. The radius of the posterior surface section 106 can be in the range of 12 mm to 40 mm, 15 mm to 34 mm, 20 mm to 28 mm, or other ranges.

Still referring to FIG. 10, the first curved surface section 94 transitions to an anterior surface section 110 (which includes label I) of the external surface 90 of the medial or lateral femoral condyle 34, 36. The anterior surface section 110 of the external surface 90 of the medial or lateral femoral condyle 34, 36 has a shape at least partially defined by an anterior section radius 112 having a length greater than the length of the radius 96 from the medial or lateral femoral condyle 34, 36 IsoHeight Point 98 to the points on the first curved surface section 94 of the external surface 90 of the medial or lateral femoral condyle 34, 36. The anterior surface section 110 is configured to articulate with the bearing surface 92 of the tibial component along a hyper-extension flexion range of less than 0°. Specifically, the anterior surface section 110 coordinates with an anterior tibial lip 93 to provide hyper-extension stability. The anterior section radius 112 of the anterior surface section 110 is greater than the first curved surface section 94 which allows for enhanced stability during hyperextension while maintaining constant contact of the femoral component 76 and the tibial component to maintain constant condylar height along an entire flexion path of the knee. The radius of the anterior surface section 110 can be in the range of 12 mm to 40 mm, 15 mm to 34 mm, 20 mm to 28 mm, or other ranges.

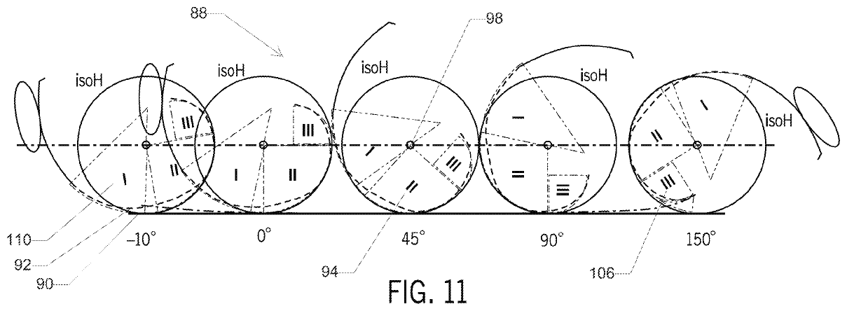

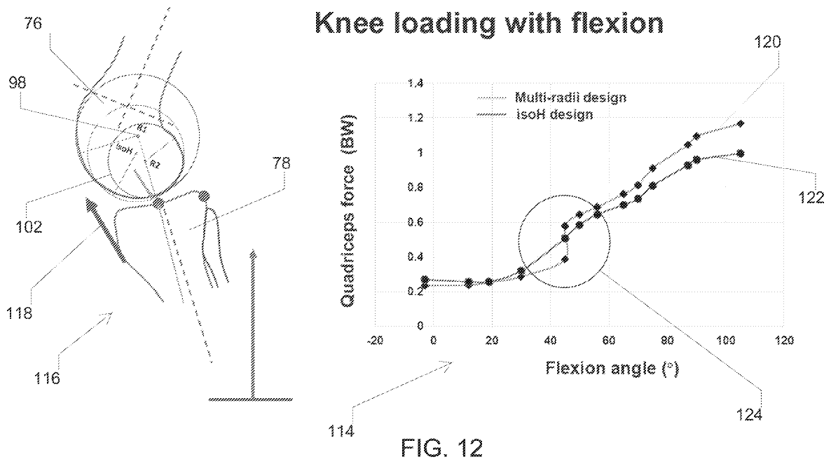

Referring now to FIG. 11, an illustration of the femoral condyle 88 including the three curved surface sections of FIG. 10 travelling through a full range of knee flexion angles is illustrated. As previously discussed, the anterior surface section 110 articulates with the bearing surface 92 of the tibial component 90 at angles of hyper-extension, i.e., less than 0° flexion, the first curved surface section 94 articulates with the bearing surface 92 of the tibial component at angles of mid-flexion, i.e., between 0° and 90° or larger, and the posterior surface section 106 articulates with the bearing surface 92 of the tibial component at angles of high-flexion, i.e., greater than 90°. As illustrated, constant contact with the bearing surface 92 of the tibial component is achieved through the specific radius of curvature of each section, and the IsoHeight Point 98 maintains a constant height throughout the full range of flexion angles. Thus, one advantage of the present disclosure is maintaining a gap balance along the entire flexion path rather than at only 0° or 90°. Additionally, matching the external femoral surfaces, i.e., the first curved surface section 94, the posterior surface section 106, and the anterior surface section 110, to the bearing surface 92 of the tibial component maintains constant condylar height during knee flexion. Maintaining constant contact also eliminates abrupt rotational or articulation velocity changes as a result of uneven surface contact between the femoral and tibial component 76, 78 associated with traditional knee prostheses.

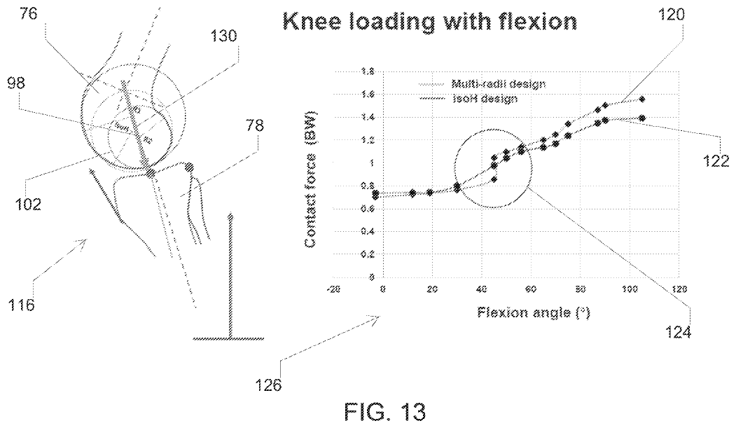

Referring now to FIG. 12, a chart 114 including quadriceps force loading on a knee during a flexion range is illustrated. FIG. 12 also includes a free body diagram 116 that illustrates the direction of the quadriceps force as indicated by arrow 118 in relation to a femoral component 76 and a tibial component 78 of a prosthesis including an IsoHeight Point 98 and circle 102. The quadriceps force 118 experienced on the tibial component 78 during knee flexion was recorded for both an IsoHeight design prosthesis and a multi-radii design prosthesis as indicated by lines 122 and 120, respectively. As illustrated, use of the IsoHeight design prosthesis 122 resulted in a much less severe quadriceps force 118 increase during a mid-flexion range 124 in comparison to the abrupt quadriceps force 118 increase of the multi-radii design prosthesis 120. As a result, another advantage of the IsoHeight prosthesis 122 over other designs is decreased abrupt stress changes on the knee joint and quadricep muscle during flexion.

Referring now to FIG. 13, a chart 126 including contact force loading on a knee during a flexion range is illustrated. FIG. 13 also includes a free body diagram 128 that illustrates the direction of the contact force as indicated by arrow 130 in relation to a femoral component 76 and tibial component 78 of a prosthesis including an IsoHeight Point 98 and circle 102. The contact force 130 experienced on the tibial component 78 during knee flexion was recorded for both the IsoHeight design prosthesis 122 and the multi-radii design prosthesis 120 as discussed for FIG. 12. As illustrated, use of the IsoHeight design prosthesis 122 resulted in a much less severe contact force 130 increase during the mid-flexion range 124 in comparison to the abrupt contact force 130 increase of the multi-radii design prosthesis 120.

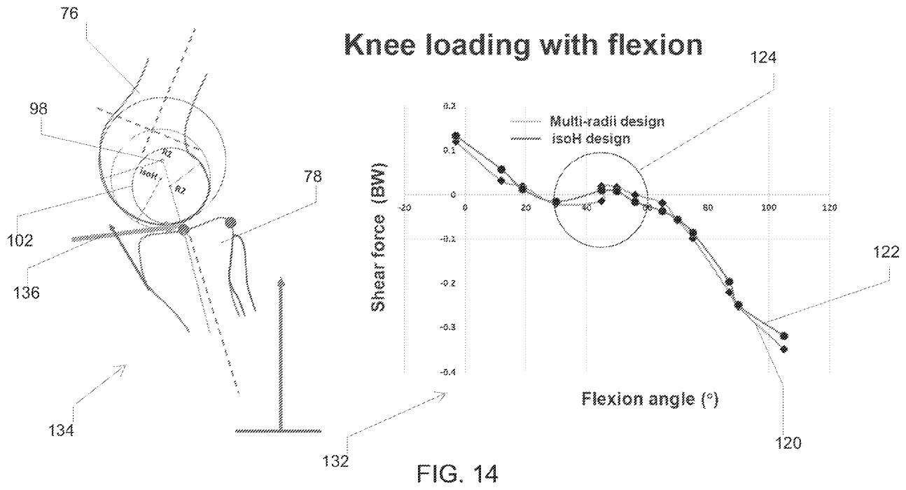

Referring now to FIG. 14, a chart 132 including shear force loading on a knee during a flexion range is illustrated. FIG. 13 also includes a free body diagram 134 that illustrates the direction of the shear force as indicated by arrow 136 in relation to a femoral component 76 and a tibial component 78 of a prosthesis including an IsoHeight Point 98 and circle 102. The shear force experienced on the tibial component 78 during knee flexion was recorded for both the IsoHeight design prosthesis 122 and the multi-radii design prosthesis 120 as discussed for FIGS. 12 and 13. As illustrated, use of the IsoHeight design prosthesis 122 resulted in a slightly less severe shear force 136 increase during the mid-flexion range 124 in comparison to the abrupt shear force 136 increase of the multi-radii design prosthesis 120.

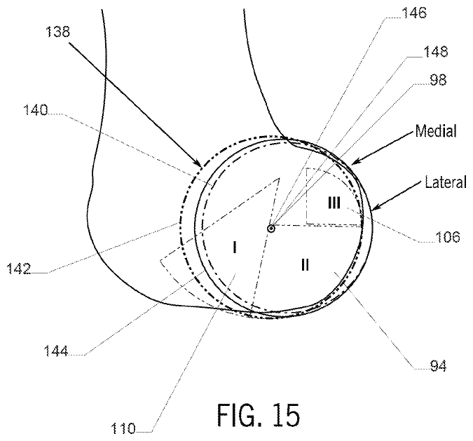

Referring now to FIG. 15, a side view of different circles of rotation for a femoral component overlayed on a native human femur is illustrated. A medial IsoHeight circle 138 of rotation, lateral IsoHeight circle 144 rotation, medial GCA circle of rotation 140, and lateral GCA circle of rotation 140 are layered over the native femur 46. An IsoHeight Point 98, a medial GCA point 146, and lateral GCA point 148 are also located at the center of their respective circles of rotation. The first curved surface section 94, posterior surface section 106, and anterior surface section 110 are sized for the lateral IsoHeight circle 144. As depicted, the medial and lateral IsoHeight circles 138, 144 of rotation are asymmetric of each other and are unique for each knee. The IsoHeight Point determination method as previously discussed provides for customization ability for each subject. As such, another advantage of the present disclosure provides increased specificity and ease of pre-operative planning and femoral or tibial cutting methods, i.e., manual cutting, navigational cutting, or robotic cutting.

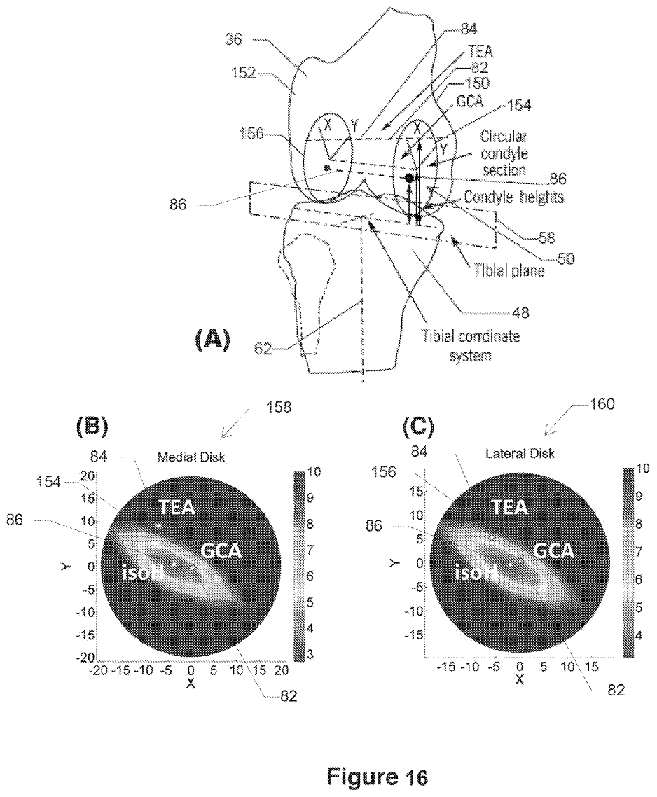

Referring now to panel (A) of FIG. 16, a perspective view of a 3D model of a 3D knee model and coordinate systems used to measure femoral condyle heights during knee flexion are illustrated. The model further includes a femur 46 and a tibia 48, and an example tibial coordinate system is shown. A tibial plane 58 is oriented within 0° to 10° of normal through a longitudinal axis 62 of the tibia 48, preferably within 5° to 7° of normal through the longitudinal axis 62 of the tibia 48. The condylar height 64 is defined as a distance measured between the tibial plane 58 and each of the virtual anatomic location points 50 along a direction normal to the tibial plane 58 for each point 50. The femur 46 has a medial side 150 and a lateral side 152. The medial side 150 has a medial femoral condyle disk 154, and the lateral side 152 has lateral femoral condyle disk 156 through which TEA 84 and GCA 82 both extend. An IsoHeight axis Point 86 is included on the medial femoral condyle disk 154. Condylar heights 64 for each axis are measured from a point on the tibial plane 58 to the TEA 84, GCA 82, and IsoHeight axis 86, respectively, along a direction perpendicular to the tibial plane 58 of the tibia 48.

Referring now to panel (B) of FIG. 16, an example chart 158 including locations of the TEA 84, GCA 82, and IsoHeight axis points on the medial femoral condyle disk 154 in the sagittal plane is illustrated. The IsoHeight axis Point 86 experienced the least amount of vertical and horizontal length change and is located at the center of rotation of the femur 46. The TEA point 84 experienced a ˜9 mm increase in vertical height and a ˜−5 mm decrease in horizontal length. While the GCA point 82 only slightly decreased in height, it experienced a ˜3 mm increase in length. The IsoHeight axis Point 86 experienced less than ˜1 mm change in both vertical height and horizontal length, thereby demonstrating the advantages of using the IsoHeight axis 86 in prosthesis design for maintaining constant condyle height during rotation in comparison to using the TEA 84 or GCA 82.

Referring now to panel (C) of FIG. 16, an example chart 160 including locations of the TEA 84, GCA 82, and IsoHeight axis 86 points on the lateral femoral condyle disk 156 in the sagittal plane is illustrated. The IsoHeight axis Point 86 experienced the least amount of vertical and horizontal length change and is located at the center of rotation. The TEA point 84 experienced a ˜7 mm increase in vertical height and a ˜−4 mm decrease in horizontal length. The GCA point 82 experienced a ˜1 mm decrease in height and a 2 mm increase in length. The IsoHeight axis Point 86 experienced less than ˜1 mm change in both vertical height and horizontal length, thereby demonstrating the advantages of using the IsoHeight axis 86 in prosthesis design for maintaining constant condyle height during rotation in comparison to using the TEA 84 or GCA 82. In some embodiments, the IsoHeight axis Point 86 for the medial femoral condyle disk 154 and lateral femoral condyle disk 156 is a different point, as depicted in panels (B) and (C) of FIG. 16.

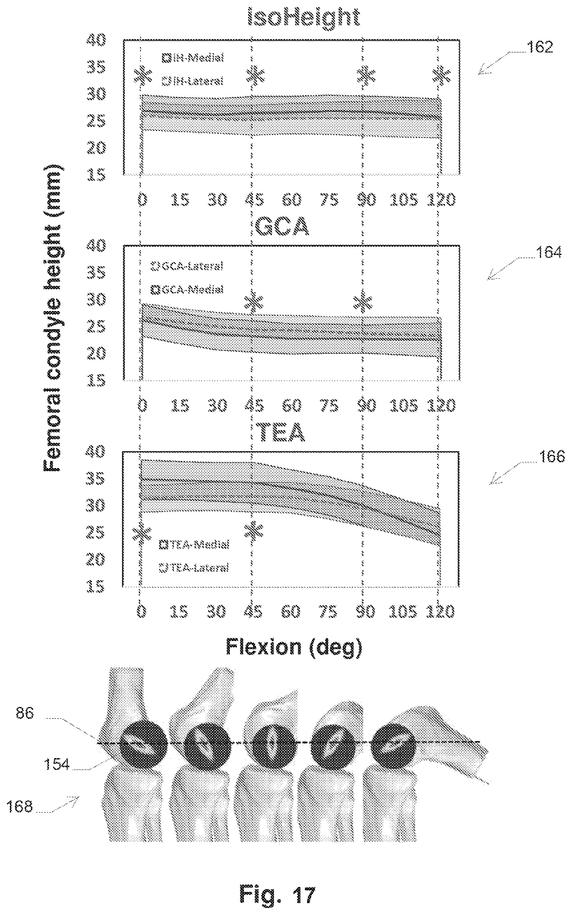

Referring now to FIG. 17, a comparison of medial and lateral femoral condyle heights measured during knee flexion using the TEA, GCA, and IsoHeight axis 86 is illustrated. In an IsoHeight chart 162, the IsoHeight medial and lateral femoral condyle height lines are shown to remain relatively constant along the flexion path with respect to their initial height measurement, whereas a TEA chart 164 and a GCA chart 166 exhibit pronounced height changes through the range of knee flexion. An example rotation path of the knee 168 including the medial femoral condyle disk 154 is also illustrated in FIG. 17.

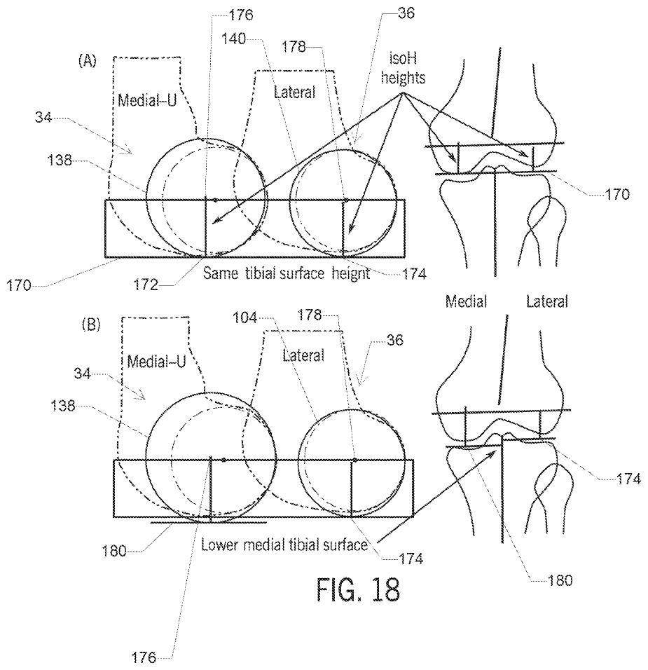

Referring now to panel (A) of FIG. 18, a side of view of a medial and a lateral femoral condyle 34, 36 that share an even tibial surface height 170 is illustrated, according to some aspects of the present disclosure. The tibial surface height 170 is defined as the condylar height 64 as discussed for FIG. 7. In some embodiments, the bearing surface 92 of the tibia 48 includes a medial tibial surface 172 and a lateral tibial surface 174 having the same height which is defined as the even tibial surface height 170. The medial and lateral tibial surfaces 172, 174 also each have an articular surface substantially asymmetric with each other for at least some portions thereof. For the case of an even medial and lateral tibial surface 172, 174 a medial IsoHeight circle 138 is drawn with a radius equal to the distance from a medial IsoHeight center, i.e., a medial IsoHeight Point 176, to the tibial surface 170, and a lateral IsoHeight circle 140 is drawn with a radius equal to the distance from a lateral IsoHeight center, i.e., a lateral IsoHeight Point 178, to the tibial surface 170. The medial and lateral IsoHeight circles 138, 140 form the main articular surfaces of the medial and lateral femoral condyles. The radius of each the medial and lateral IsoHeight circles 138, 140 defines the corresponding medial and lateral femoral condylar heights.

Referring now to panel (B) of FIG. 18, a side of view of a medial and a lateral femoral condyle 34, 36 with uneven tibial surface heights is shown, according to some aspects of the present disclosure. In some aspects, a medial tibial surface 180 can be deeper than a lateral tibial surface 174. An uneven tibial surface height design simulates the physiological uneven medial and lateral tibial surface heights 180, 174 in native human knees. For the case of uneven medial and lateral tibial surfaces 180, 174, a medial IsoHeight circle 138 is drawn with a radius equal to the distance from a medial IsoHeight center, i.e., a medial IsoHeight Point 176, to the deeper medial tibial surface 180, and a lateral IsoHeight circle 140 102 is drawn with a radius equal to the distance from a lateral IsoHeight center, i.e., a lateral IsoHeight Point 178, to the lateral tibial surface 174. The radius of each the medial and lateral IsoHeight circles 138, 140 defines the corresponding medial and lateral femoral condylar heights.

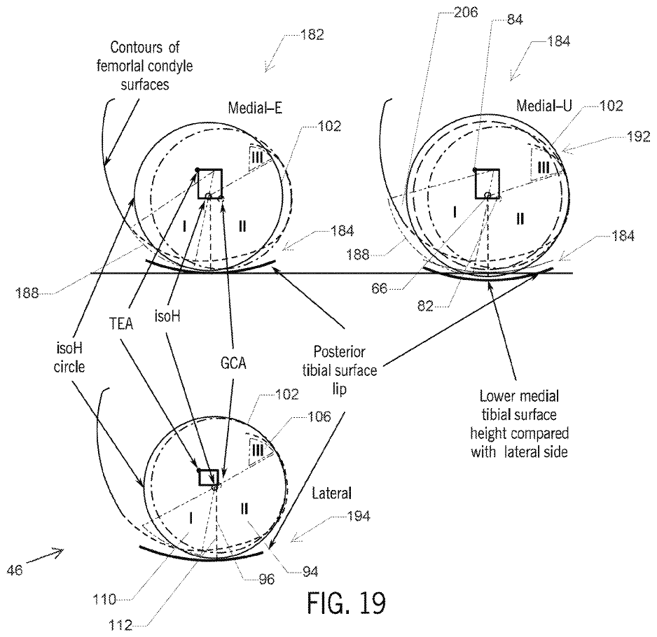

Referring now to FIG. 19, a side view of even and uneven medial 182, 184 and lateral femoral condyles 36 including diagrams of curved surface sections is illustrated. For both the even and uneven tibial surface cases, at low hyperextension angles, the IsoHeight circle 102 needs to connect an anterior portion/trochlear groove 188 of the femur 46 to closely maintain the general anatomy of the knee. Therefore, a first curved surface section 94 as discussed for FIG. 10 transitions to an anterior surface section 110. In some aspects, the anterior surface section 110 has an anterior section radius 112 that is greater than a radius 96 of the first curved surface section 94. To facilitate high knee flexion, a posterior surface section 106 as discussed for FIG. 10 extends from the first curved surface section 94 to round up the posterior-proximal end 106 of the femoral condyles 182, 184, 36. To maintain femoral condyle height at high flexion, the posterior surface section 106 articulates with and is supplemented by a posterior tibial lip 194 of the bearing surface 92 of the tibia 48. A GCA point 82 and a TEA point 84 are included at different locations on the IsoHeight circle 102 to demonstrate the difference in location with the IsoHeight Point 66.

For both the even and uneven tibial surface cases, at high flexion angles, the IsoHeight circle 102 is stopped at a location close to the posterior-proximal end 192 of the femoral condyles 182, 184, 36 to be consistent with the native knee anatomy. To facilitate high knee flexion, a posterior surface section 106 as discussed for FIG. 10 extends from the first curved surface section 94 to round up the posterior-proximal end 192 of the femoral condyles 182, 184, 36. To maintain femoral condyle height at high flexion, the posterior surface section 106 articulates with and is supplemented by a posterior tibial lip 194 of the bearing surface 92 of the tibia 48. A GCA point 82 and a TEA point 84 are included at different locations on the IsoHeight circle 102 to demonstrate the difference in location with the IsoHeight Point 66.

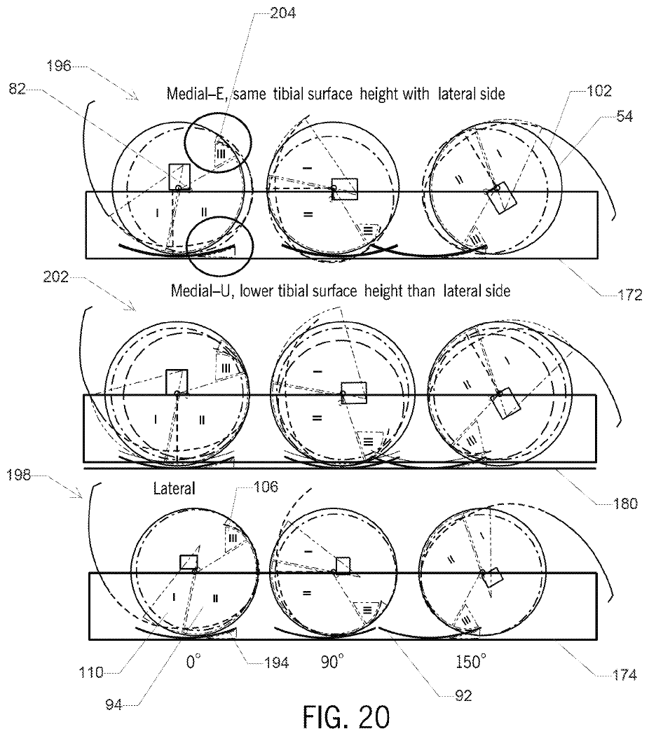

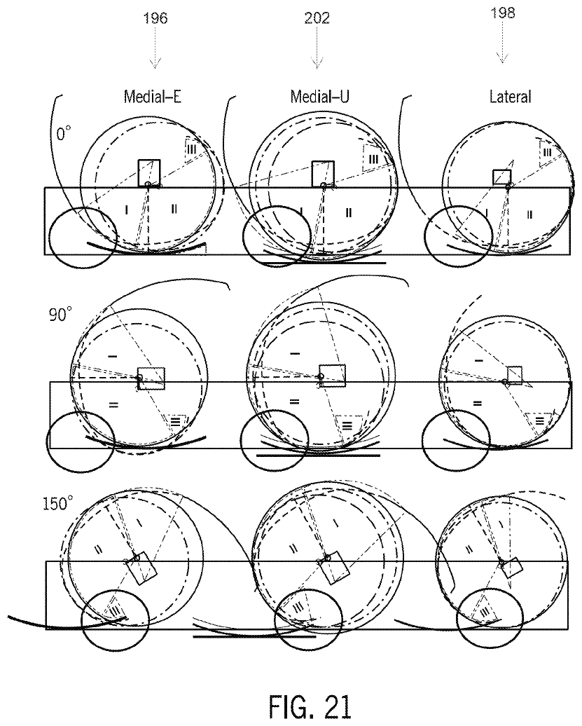

Referring now to FIG. 20, an array of the femoral condyles as depicted in FIG. 19 being rotated through a range of flexion angles (e.g., 0°, 90°, 150°) are illustrated. In the top panel of FIG. 20, an even medial femoral condyle path 196 (Medial-E) includes an even medial tibial surface 172 with the lateral tibial surface 174 of a lateral femoral condyle path 198 (Lateral) in the bottom panel of FIG. 20. In the middle panel of FIG. 20, an uneven medial femoral condyle path 202 (Medial-U) includes an uneven medial tibial surface 180 that is deeper than the lateral tibial surface 174 of the lateral femoral condyle path 198 (Lateral) in the bottom panel of FIG. 20. The IsoHeight circle 102 includes the three surface sections, i.e., a first curved surface section 94, a posterior surface section 106, and an anterior surface section 110, and the posterior surface section 106 articulates with a posterior tibial lip 194 of the bearing surface 92 as the IsoHeight circle 102 rotates to a deep flexion angle. A space between an outer surface 204 of the posterior surface section 106 and an outer circumference 54 of the IsoHeight circle 102 is intentionally designed to match a shape of the bearing surface 92 in order to maintain constant articulation heights of the medial and lateral femoral condyles with the bearing surface 92. In some aspects, the bearing surface 92 is adjusted to match the shape of the medial and lateral femoral condyles. The IsoHeight Point 66 remains at the center of rotation of the IsoHeight circle 102 throughout flexion while the GCA point 82 does not. Another array of the medial and lateral femoral flexion paths (e.g., 0°, 90°, 150°) and rotation views of FIG. 20 are illustrated in FIG. 21.

Thus, the present invention provides an advantage in easing gap/soft tissue balance along a flexion path of the knee, improving mid-range instability, reducing overstuffing at high flexion, enhancing natural knee feeling, and improving surgery longevity. The systems and methods described herein provide stability to the knee at low flexion, mid-flexion, and high flexion.

The present invention provides another advantage in maintaining the native femoral condyle height along the flexion path of the knee to maintain the medial and lateral compartment balance of the knee. To keep the physiological condyle height, the tensions of the surrounding soft tissues such as medial and lateral collateral ligaments are also maintained.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

1. A knee joint prosthesis comprising:
a femoral component including an internal non-articulating bone-engaging surface configured to be connected to a resected distal end of a femur, the femoral component including a medial femoral condyle having an external surface configured to articulate with a bearing surface of a tibial component, the external surface of the medial femoral condyle including a first curved surface section,
wherein the first curved surface section of the external surface of the medial femoral condyle has an arcuate shape defined by a radius from a medial femoral condyle IsoHeight Point to points on the first curved surface section of the external surface of the medial femoral condyle.

2. The knee joint prosthesis of claim 1 wherein:
the femoral component further includes a lateral femoral condyle having an external surface configured to articulate with a bearing surface of a tibial component, the external surface of the lateral femoral condyle including a first curved surface section of the external surface of the lateral femoral condyle, and
the first curved surface section of the external surface of the lateral femoral condyle has an arcuate shape defined by a radius from a lateral femoral condyle IsoHeight Point to points on the first curved surface section of the external surface of the lateral femoral condyle.

3. A knee joint prosthesis comprising:
a femoral component including an internal non-articulating bone-engaging surface configured to be connected to a resected distal end of a femur, the femoral component including a lateral femoral condyle having an external surface configured to articulate with a bearing surface of a tibial component, the external surface of the lateral femoral condyle including a first curved surface section of the external surface of the lateral femoral condyle,
wherein the first curved surface section of the external surface of the lateral femoral condyle has an arcuate shape defined by a radius from a lateral femoral condyle IsoHeight Point to points on the first curved surface section of the external surface of the lateral femoral condyle.

4. The knee joint prosthesis of claim 3 wherein:
the femoral component further includes a medial femoral condyle having an external surface configured to articulate with a bearing surface of a tibial component, the external surface of the medial femoral condyle including a first curved surface section, and
the first curved surface section of the external surface of the medial femoral condyle has an arcuate shape defined by a radius from a medial femoral condyle IsoHeight Point to points on the first curved surface section of the external surface of the medial femoral condyle.

5. The knee joint prosthesis of claim 2 wherein:
the first curved surface section of the external surface of the medial femoral condyle transitions to a posterior surface section of the external surface of the medial femoral condyle, the posterior surface section of the external surface of the medial femoral condyle having a shape at least partially defined by a posterior section radius having a length less than a length of the radius from the medial femoral condyle IsoHeight Point to the points on the first curved surface section of the external surface of the medial femoral condyle.

6. The knee joint prosthesis of claim 2 wherein:
the first curved surface section of the external surface of the medial femoral condyle transitions to an anterior surface section of the external surface of the medial femoral condyle, the anterior surface section of the external surface of the medial femoral condyle having a shape at least partially defined by an anterior section radius having a length greater than the length of the radius from the medial femoral condyle IsoHeight Point to the points on the first curved surface section of the external surface of the medial femoral condyle.

7. The knee joint prosthesis of claim 2 wherein:
the first curved surface section of the external surface of the medial femoral condyle transitions to a posterior surface section of the external surface of the medial femoral condyle, the posterior surface section of the external surface of the medial femoral condyle having a shape at least partially defined by a posterior section radius having a length less than a length of the radius from the medial femoral condyle IsoHeight Point to the points on the first curved surface section of the external surface of the medial femoral condyle, and
the first curved surface section of the external surface of the medial femoral condyle transitions to an anterior surface section of the external surface of the medial femoral condyle, the anterior surface section of the external surface of the medial femoral condyle having a shape at least partially defined by an anterior section radius having a length greater than the length of the radius from the medial femoral condyle IsoHeight Point to the points on the first curved surface section of the external surface of the medial femoral condyle.

8. The knee joint prosthesis of claim 2 wherein:
the first curved surface section of the external surface of the lateral femoral condyle transitions to a posterior surface section of the external surface of the lateral femoral condyle, the posterior surface section of the external surface of the lateral femoral condyle having a shape at least partially defined by a posterior section radius having a length less than a length of the radius from the lateral femoral condyle IsoHeight Point to the points on the first curved surface section of the external surface of the lateral femoral condyle.

9. The knee joint prosthesis of claim 2 wherein:
the first curved surface section of the external surface of the lateral femoral condyle transitions to an anterior surface section of the external surface of the lateral femoral condyle, the anterior surface section of the external surface of the lateral femoral condyle having a shape at least partially defined by an anterior section radius having a length greater than the length of the radius from the lateral femoral condyle IsoHeight Point to the points on the first curved surface section of the external surface of the lateral femoral condyle.

10. The knee joint prosthesis of claim 2 wherein:
the first curved surface section of the external surface of the lateral femoral condyle transitions to a posterior surface section of the external surface of the lateral femoral condyle, the posterior surface section of the external surface of the lateral femoral condyle having a shape at least partially defined by a posterior section radius having a length less than a length of the radius from the lateral femoral condyle IsoHeight Point to the points on the first curved surface section of the external surface of the lateral femoral condyle, and
the first curved surface section of the external surface of the lateral femoral condyle transitions to an anterior surface section of the external surface of the lateral femoral condyle, the anterior surface section of the external surface of the lateral femoral condyle having a shape at least partially defined by an anterior section radius having a length greater than the length of the radius from the lateral femoral condyle IsoHeight Point to the points on the first curved surface section of the external surface of the lateral femoral condyle.

11. The knee joint prosthesis of claim 2 wherein:
the knee joint prosthesis includes the tibial component, and
the femoral component and the tibial component articulate so as to maintain substantially constant condylar heights at a medial side and a lateral side of a knee through a full range of flexion.

12. The knee joint prosthesis of claim 11 wherein:
heights of the lateral femoral condyle and the medial femoral condyle are different to articulate on corresponding surfaces of the tibial component.

13. The knee joint prosthesis of claim 2 wherein:
a substantially constant condyle height at a medial side and/or a lateral side of a knee is achieved by minimizing vertical height changes of the medial femoral condyle IsoHeight Point or the lateral femoral condyle IsoHeight Point through coordinated articulation of anterior-posterior tibial slopes with the medial femoral condyle or the lateral femoral condyle.

14. The knee joint prosthesis of claim 2 wherein:
the medial femoral condyle and the lateral femoral condyle are in an asymmetric configuration.

15. The knee joint prosthesis of claim 2 wherein:
the knee joint prosthesis includes the tibial component, and
a surface of the tibial component includes both a medial and lateral surface having the same height and each having an articular surface substantially asymmetric with each other for at least portions thereof.

16. The knee joint prosthesis of claim 2 wherein:
the knee joint prosthesis includes the tibial component, and
a surface of the tibial component includes both a medial and lateral surface having a deeper medial tibial surface height than a lateral tibial surface height and each having an articular surface substantially asymmetric with each other for at least portions thereof.

17. The knee joint prosthesis of claim 2, further comprising design elements for posterior stabilization, cruciate retaining, bi-cruciate stabilization and/or bi-cruciate retaining functionality.

18. The knee joint prosthesis of claim 2 wherein:
the prosthesis exhibits no mid-range articulation transition zone to at least minimize mid-range instability.

19. The knee joint prosthesis of claim 2 wherein:
no sudden change in knee loading is promoted through a full range of flexion.

20. The knee joint prosthesis of claim 19 wherein:
the full range of flexion comprises from approximately −10 degrees to 130 degrees or larger.

21. The knee joint prosthesis of claim 2 wherein:
the prosthesis comprises a material selected from the group consisting of 3D printable polymers, polyethylene, cross-link polyethylene, polyether ether ketone (PEEK), titanium, titanium alloy, tantalum, cobalt chrome alloy, stainless steel, and ceramics.

22. The knee joint prosthesis of claim 2 wherein:
the prosthesis maintains medial and lateral ligament tension balances through a full range of motion without overstretching soft-tissue in a knee.

23. A method comprising:
determining from anatomical and kinematic analysis an appropriate medial femoral condyle IsoHeight Point and an appropriate lateral femoral condyle IsoHeight Point for a subject's knee; and
selecting or fabricating for implantation a knee prosthesis according to claim 2.

24. The method of claim 23, wherein determining comprises acquiring images of the subject's knee with or without motion thereof and calculating from the acquired images appropriate dimension specifications for the knee joint prosthesis.

25. The method of claim 23, further comprising surgically implanting the knee joint prosthesis into the subject's knee through manual, navigational, or robotic methods.

26. A method for manufacturing a knee joint prosthesis, the method comprising:
forming a femoral component including an internal non-articulating bone-engaging surface configured to be connected to a resected distal end of a femur,
wherein the femoral component includes a medial femoral condyle having an external surface configured to articulate with a bearing surface of a tibial component, the external surface of the medial femoral condyle including a first curved surface section, wherein the first curved surface section of the external surface of the medial femoral condyle has an arcuate shape defined by a radius from a medial femoral condyle IsoHeight Point to points on the first curved surface section of the external surface of the medial femoral condyle, and
wherein the femoral component further includes a lateral femoral condyle having an external surface configured to articulate with a bearing surface of a tibial component, the external surface of the lateral femoral condyle including a first curved surface section of the external surface of the lateral femoral condyle, wherein the first curved surface section of the external surface of the lateral femoral condyle has an arcuate shape defined by a radius from a lateral femoral condyle IsoHeight Point to points on the first curved surface section of the external surface of the lateral femoral condyle.

27. The method of claim 26 wherein:
the medial femoral condyle IsoHeight Point is a medial anatomic location of the medial femoral condyle determined by: (i) creating from medical images, a model of a knee joint of a subject including a femur and a tibia having ends to be replaced by a knee joint prosthesis, (ii) orienting a longitudinal axis on the tibia of the model, (iii) placing a tibial plane on the model at a proximal end of the tibia of the model, the tibial plane being oriented within 0° to 10° of normal through the longitudinal axis of the tibia of the model, (iv) rotating the femur of the model through a full range of flexion, (v) choosing the medial anatomic location to be a point in which a least amount of change of condyle height occurs throughout the full range of flexion from an initial height from the tibial plane, and
the lateral femoral condyle IsoHeight Point is a lateral anatomic location of the lateral femoral condyle determined by: (i) creating from medical images, a model of a knee joint of a subject including a femur and a tibia having ends to be replaced by a knee joint prosthesis, (ii) orienting a longitudinal axis on the tibia of the model, (iii) placing a tibial plane on the model at a proximal end of the tibia of the model, the tibial plane being oriented within 0° to 10° of normal through the longitudinal axis of the tibia of the model, (iv) rotating the femur of the model through a full range of flexion, (v) choosing the lateral anatomic location to be a point in which a least amount of change of condyle height occurs throughout the full range of flexion from an initial height from the tibial plane.
